(19) United States
(12) Plant Patent Application Publication
(10) Pub. No.: US 2005/0216985 P1
(43) Pub. Date: Sep. 29, 2005

(54) FICUS PLANT NAMED 'BLACK DIAMOND'

(50) FICUS BENJAMINA; FICUS

(75) Inventor: Jan van Geest, s'Gravenzande (NL)

Correspondence Address: FOLEY AND LARDNER;SUITE 500, 3000 K STREET NW, WASHINGTON, DC, 20007, US

(73) Assignee: Plantenkwekerij J. van Geest, B.V.

(21) Appl. No.: 10/806,389

(22) Filed: Mar. 23, 2004

(51) Int. Cl. A01H 5/00

(52) U.S. Cl. PLT/211

(57) ABSTRACT

A new and distinct variety of Ficus benjamina designated ‘Black Diamond’, particularly characterized by variegated leaves with very dark green foliage with amorphous, irregularly shaped and irregularly patterned spots of yellow green along the midrib of the leaf. ‘Black Diamond’ is heavily branching with an upright growth habit.

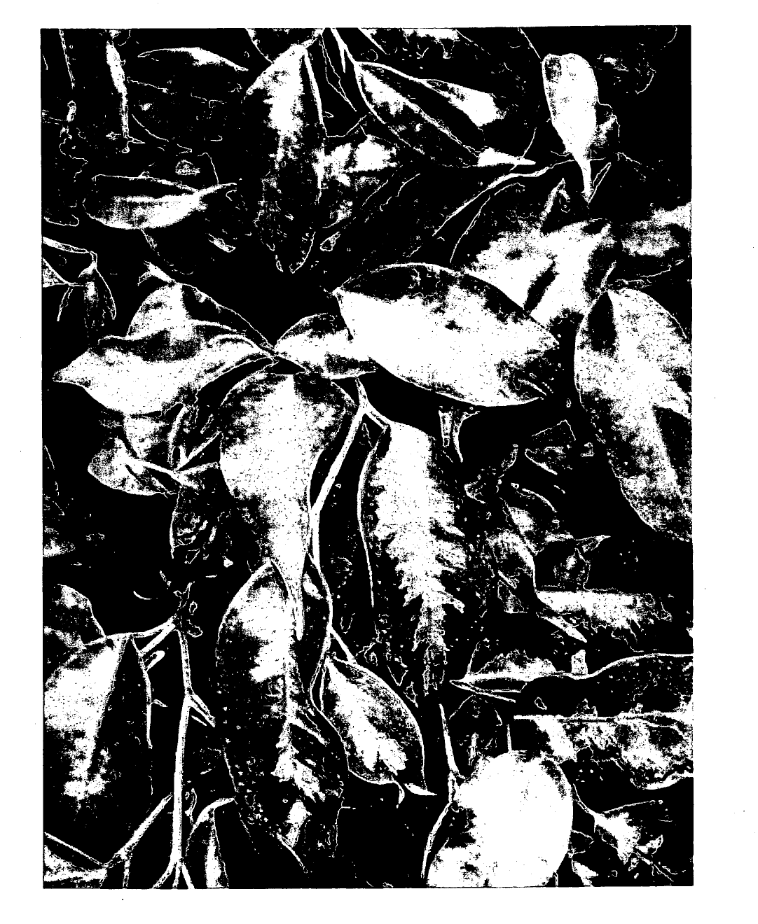

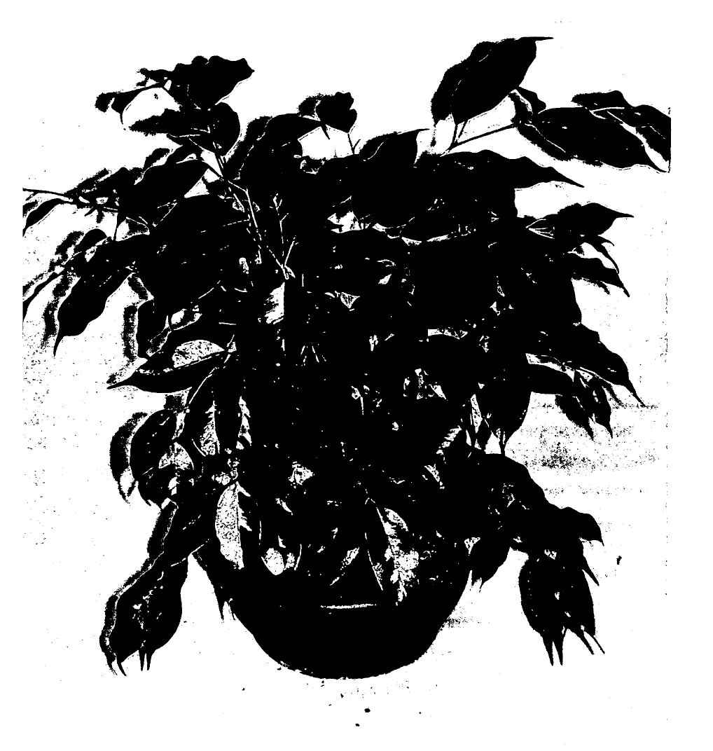

### LATIN NAME OF THE GENUS AND SPECIES OF THE PLANT CLAIMED

Ficus benjamina

### VARIETY DENOMINATION

Black Diamond

### BACKGROUND OF THE INVENTION

The present invention relates to a new and distinct cultivar of Ficus benjamina. The varietal denomination of the new cultivar is ‘Black Diamond’. The new variety was discovered as a mutation in a controlled planting of Ficus benjamina ‘Midnight’ in a greenhouse in s'Gravenzande, The Netherlands in June, 2001.

### SUMMARY OF THE INVENTION

The new variety is a mutation of the Ficus variety ‘Midnight’ (U.S. Plant Pat. No. 9,856), and was selected by the inventor, Jan van Geest. The mutation was selected among plants of ‘Midnight’ which were grown in a controlled environment in a glasshouse in s'Gravenzande, The Netherlands in June, 2001. The new variety was first asexually propagated by vegetative cutting in August 2001, in s'Gravenzande, The Netherlands. Asexual reproduction through succeeding generations has established that the combination of characteristics as herein disclosed for the new cultivar are firmly fixed and retained through successive generations of asexual reproduction.

In comparison to the parent plant, ‘Midnight’, the leaf color of ‘Black Diamond’ is variegated. The leaves of ‘Black Diamond’ are very dark green (near 137 A, new foliage, upper side), with an amorphous, irregularly shaped and irregularly patterned spot of yellow-green (RHS 144A) located along the midrib of the leaf. The leaves of ‘Midnight’ by comparison are 137B (new foliage, upper side). In addition, ‘Black Diamond’ varies from the parent plant in leaf and petiole size. The leaves of ‘Black Diamond’ are narrower, 3.5 cm, as compared to 4 cm leaf width of ‘Midnight’, and shorter, 9 cm, as compared to 11 cm long leaves of ‘Midnight’. The petioles of ‘Black Diamond’ are shorter, approximately 5 mm in length, as compared to 18 mm long petioles of ‘Midnight’.

### BRIEF DESCRIPTION OF THE INVENTION

The following traits have been repeatedly observed and are determined to be basic characteristics of ‘Black Diamond’ which in combination distinguish this Ficus as a new and distinct cultivar from parent cultivar ‘Midnight’ and other Ficus cultivars:
1. Variegated colored leaves, having a dark green colored leaf with a yellow-green spot;
2. irregularly shaped leaves; and
3. irregular patterned spots of yellow-green located along the midrib of the leaf
4. heavily branching, upright growth habit.

### BRIEF DESCRIPTION OF THE DRAWINGS

The accompanying photographic drawings show a specimen of the new cultivar as true to color as is reasonably possible. The first photographic drawing is a side view of ‘Black Diamond’ in its entirety showing the growth habit. The second photographic drawing is a close up of the foliage of ‘Black Diamond’. Phenotypic expression may vary depending on light, environmental and cultural conditions.

### DETAILED DESCRIPTION OF THE NEW VARIETY

‘Black Diamond’ has not been observed under all possible environmental conditions. The phenotypic expression may vary with variations in environment such as temperature, light intensity, day length and cultural conditions, without a change in the genotype of the plant.

The following observations and descriptions are of a plant grown in a shade house covered with plastic wrap providing 73% shade in Homestead, Fla., during the months of January through July, 2003. Color references are to The Royal Horticultural Society Colour Chart (RHS). The color references are as approximate as possible as color depends to a degree on horticultural practices such as light level and fertilization rates.
Plant:
   Form.—Medium compact, somewhat weeping, upwardly branching, ascending regularly and richly branching.
   Growth.—Lateral branches are approximately at a 30 to 45 degree angle to the trunk, but slightly weeping at the top; maximum height 1 meter.
   Growth habit.—Heavily branching, upright.
Foliage:
   Size.—About 3.5 cm wide by 9 cm long (to leaf tip).
   Color.—New foliage: upper side green near 137 A, with the spot along the midrib of leaf being closest to yellow-green 144A; underside 138A with the spot along the midrib closest to 139C. Old foliage: upper side green 131 with the spot along the midrib near yellow-green 144A; underside near 137C with the spot along the midrib near 138D.
   Shape.—Ovate with acuminate leaf tip slightly coarsely sinuate and glabrous.
   Texture.—Smooth, leathery, glossy, underside smooth, matte.
   Veination.—Single mid-rib vein running from petiole to leaf tip, convex on underside, light green near 145B.
   Margin.—Entire, slightly undulate.
   Serration.—None.
   Stipules.—Leaves are glabrous.
   Petiole.—Closest to 144A; about 5 mm long.
   Disease resistance.—No particular claim observation made.
   Rachis.—None.
Wood:
   New shoots.—Color near 144A; smooth.
   Old wood.—Color near 197B; no flowers or fruit observed to date.
   Internode spacing.—About 30 mm.
   Preferred growing conditions.—20 degrees C. to 32 degrees C.
   Reproductive organs.—None observed.
   Temperature range.—To 2 degrees C.

1. A new and distinct variety of Ficus benjamina designated ‘Black Diamond’, as illustrated and described herein.
